(19) United States
(12) Patent Application Publication
(10) Pub. No.: US 2009/0143237 A1
(43) Pub. Date: Jun. 4, 2009

(54) Methods, kits and compositions pertaining to fluorescence quenching using pna probes

(75) Inventors: Henrik Stender, Gentofte (DK); Thais T. Johansen, Vedbaek (DK)

Correspondence Address: EDWARDS ANGELL PALMER & DODGE LLP, P.O. BOX 55874, BOSTON, MA, 02205, US

(73) Assignee: AdvanDx, Inc., Woburn, MA (US)

(21) Appl. No.: 10/592,961

(22) PCT Filed: Mar. 18, 2005
(86) PCT No.: PCT/US05/09157

§ 371 Date: Sep. 8, 2008

Related U.S. Application Data
(60) Provisional application No. 60/554,516, filed on Mar. 18, 2004.

(51) Int. Cl. C40B 30/04; G01N 33/00; C40B 40/08; C12Q 1/68

(52) U.S. Cl. 506/9; 436/94; 435/6; 506/17

(57) ABSTRACT

Disclosed for instance, are methods suitable for the detection, identification and/or quantitation of nucleic acid target sequences using probes. Suitable probes include those consisting of peptide nucleic acid (PNA) or locked nucleic add (LNA) units. Binding of the probes to adjacent target sequences results in fluorescent quenching. Analysis of changes in the fluorescent signal is used to detect, identify or quantitate a target sequence in a sample. The invention is more specifically directed to methods for improving the sensitivity, specificity and/or reliability of diagnostic tests using suitable probes. It is particularly well-suited for real-time PCR and related amplification reaction.

### CROSS-REFERENCE TO RELATED APPLICATION

The present International Application claims priority to U.S. Provisional Application No. 60/554,516 as filed on Mar. 18, 2004. The disclosure of the 60/554,516 Provisional Application is incorporated herein by reference.

### FIELD OF THE INVENTION

This invention relates to a system, methods, kits and compositions suitable for the detection, identification and/or quantitation of nucleic acid target sequences using PNA probes. Binding of PNA probes to adjacent target sequences results in fluorescent quenching. Analysis of changes in the fluorescent signal is used to detect, identify or quantitate a target sequence in a sample. The invention is more specifically directed to methods, kits and compositions suitable for improving the sensitivity, specificity and/or reliability of diagnostic tests using PNA probes. The methods, kits and compositions of this invention are particularly well-suited for real-time PCR.

### BACKGROUND OF THE INVENTION

This invention is related to the field of probe based nucleic acid sequence detection, quantitation and analysis.

Nucleic acid hybridization is a fundamental physiochemical process, central to the understanding of molecular biology. Probe-based assays use hybridization for the detection, quantitation and analysis of nucleic acids. Nucleic acid probes have long been used to analyze samples from a variety of sources for the presence of nucleic acids, as well as to examine clinical conditions of interest in single cells and tissues. More recently, PNA probes have become the preferred reagents for hybridization assays.

Despite its name, Peptide Nucleic Acid (PNA) is neither a peptide nor a nucleic acid, it is not even an acid. PNA is a non-naturally occurring polyamide that can hybridize to nucleic acid (DNA and RNA) with sequence specificity (See: U.S. Pat. No. 5,539,082) and Egholm et al., Nature 365:566-568 (1993)) according to Watson-Crick base paring rules. PNAs are synthesized by adaptation of standard peptide synthesis procedures in a format, which is now commercially available. For a general review of the preparation of PNA monomers and oligomers please see: Dueholm et al., New J. Chem., 21: 19-31 (1997) or Hyrup et. al., Bioorganic & Med. Chem. 4: 5-23 (1996). Alternatively, labeled and unlabeled PNA oligomers can be purchased (Applied Biosystems, Foster City, Calif.). Despite the ability to hybridize to nucleic acid in a sequence specific manner, there are many differences between PNA probes and standard nucleic acid probes. These differences can be conveniently broken down into biological, structural, and physico-chemical differences. This non-equivalency of differing compositions is often observed in the chemical arts.

With regard to biological differences, nucleic acids, are biological materials that play a central role in the life of living species as agents of genetic transmission and expression. Their in vivo properties are fairly well understood. PNA on the other hand is a recently developed totally artificial molecule, conceived in the minds of chemists and made using synthetic organic chemistry. It has no known biological function (i.e. native (unmodified) PNA is not known to be a substrate for any polymerase, ligase, nuclease or protease).

Structurally, PNA also differs dramatically from nucleic acid. Although both can employ common nucleobases (A, C, G, T, and U), the backbones of these molecules are structurally diverse. The backbones of RNA and DNA are composed of repeating phosphodiester ribose and 2-deoxyribose units. In contrast, the backbones of the most common PNAs are composed on (aminoethyl)-glycine subunits. Additionally, in PNA the nucleobases are connected to the backbone by an additional methylene carbonyl moiety. PNA is not an acid and therefore contains no charged acidic groups such as those present in DNA and RNA. Because they lack formal charge, PNAs are generally more hydrophobic than their equivalent nucleic acid molecules. The hydrophobic character of PNA allows for the possibility of non-specific (hydrophobic/hydrophobic interactions) interactions not observed with nucleic acids. Further, PNA is achiral, providing it with the capability of adopting structural conformations the equivalent of which do not exist in the RNA/DNA realm. The unique structural features of PNA result in a polymer that is highly organized in solution, particularly for purine rich polymers (See: Dueholm et al., New J. Chem., 21: 19-31 (1997) at p. 27, col. 2, lns. 6-30). Conversely, a single stranded nucleic acid is a random coil, which exhibits very little secondary structure. Because PNA is highly organized, PNA should be more resistant to adopting alternative secondary structures (e.g. a hairpin stem and/or loop).

The physico/chemical differences between PNA and DNA or RNA are also substantial and described in more details below:

Higher Sensitivity: PNA binds stronger and faster to complementary RNA or DNA hereby facilitating the development of more rapid diagnostic tests. Where DNA probes often require over-night incubation, reactions with PNA are completed within a few hours (for example see: Thisted et al., Cell Vision 3:358-363 (1996)).
Higher Specificity: PNA probes are particularly well suited for the discrimination between closely related sequences, even single nucleotide differences. This makes PNA-based assays highly specific (for example see: Igleo, G. L. BioTechniques 27:798-808 (1999)).
Robust Assays: PNA is a synthetic molecule resistant to nucleases and proteases and thus extremely stable in prepackaged kit formats as well as during the actual assay where in contact with the sample (for example see: Demidov, Biochem. Pharmacol. 48:1310-1313 (1994)).
Novel Assay Formats: The unique properties of PNA enable the development of assay formats, which go above and beyond the possibilities of DNA probes, hereby reducing the complexity related to the performance of molecular diagnostic tests (for examples see: Stender et al., J. Microbiol. Methods 48:1-17 (2002)).
No Target Limitation: The non-charged backbone allows PNA probes to hybridize under conditions that are destabilizing to DNA and RNA. Attributes that enable PNA probes to access targets, such as highly structured rRNA and double stranded DNA, known to be inaccessible to DNA probes (See: Stephano & Hyldig-Nielsen, IBC Library Series Publication #948. International Business Communication, Southborough, Mass., pp. 19-37 (1997)).
In Situ Hybridization: The hydrophobic nature of PNA relative to DNA makes PNA probes superior for in situ hybridization assays, where the probes must penetrate the hydrophobic cell wall prior to hybridization (for example see Stender et al., Int. J. Tuberc. Lung. Dis. 3:830-837 (1999)).

In summary, because PNAs hybridize to nucleic acids with sequence specificity, PNAs are useful candidates for investigation when developing probe-based hybridization assays. However, PNA probes are not the equivalent of nucleic acid probes in structure or function.

Probe based assays are useful in the detection, identification and quantitation of nucleic acids. Nucleic acid probes have long been used to analyze samples for the presence of nucleic acid from a bacteria, fungi, virus or other organism (See for example; U.S. Pat. Nos. 4,851,330, 5,288,611, 5,567,587, 5,601,984 and 5,612,183). Probe-based assays are also useful for examining genetically based clinical conditions of interest. Despite the high specificity of PNA probes as compared to naturally occurring nucleic acid probes, it is common to encounter sequence regions where it is very difficult to design a probe which allows exclusive detection of the desired target in a particular assay format.

Polymerase chain reaction (PCR) and other target amplification technologies has since the invention in the early-1980s via numerous improvements, including both automation and novel reagents been brought to a point where target amplification and subsequent analysis can be performed as a ‘homogeneous real-time assay’. Self-reporting PNA probes are examples of novel detection reagents offering improved simplicity and specificity, which combined with the use of rDNA, a well-established phylogenetic marker, are particularly well-suited for the diagnosis of infectious diseases (Stender et al., J. Clin. Microbiol. 40:247-251 (2002)). Besides it potential within clinical pathology, this technology also offers great potential for detecting organisms in food, beverages, water, pharmaceutical products, personal care products, dairy products or environmental samples, target sequences which are specific for a genetically based disease or specific for a predisposition to a genetically based diseases, or target sequences in a forensic technique such as prenatal screening, paternity testing, identity confirmation or crime investigation.

Quenching of fluorescence signal can occur by either Fluorescence Resonance Energy Transfer “FRET” (also known as non-radiative energy transfer: See: Yaron et al., Analytical Biochemistry 95: 228-235 (1979) at p. 232, col. 1, lns. 32-39) or by non-FRET interactions (also known as radiationless energy transfer; See: Yaron et al., Analytical Biochemistry 95 at p. 229, col. 2, lns. 713). The critical distinguishing factor between FRET and non-FRET quenching is that non-FRET quenching requires short range interaction by “collision” or “contact” and therefore requires no spectral overlap between the moieties of the donor and acceptor pair (See: Yaron et al., Analytical Biochemistry 95 at p. 229, col. 1, lns. 22-42). Conversely, FRET quenching requires spectral overlap between the donor and acceptor moieties and the efficiency of quenching is directly proportional to the distance between the donor and acceptor moieties of the FRET pair (See: Yaron et al., Analytical Biochemistry 95 at p. 232, col. 1, ln. 46 to col. 2, ln. 29). Extensive reviews of fluorescent quenching phenomena are described in Clegg, R. M., Methods Enzymol., 221: 353-388 (1992) and Selvin, P. R., Methods Enzymol., 246: 300-334 (1995). Yaron et al. also suggested that the principles described therein might be applied to the hydrolysis of oligonucleotides (See: Yaron et al., Analytical Biochemistry 95 at p. 234, col. 2, lns. 14-18).

Fluorescence quenching has been utilized for the direct detection of nucleic acid target sequences without the requirement that labeled nucleic acid hybridization probes or primers be separated from the hybridization complex prior to detection (See: Livak et al. U.S. Pat. No. 5,538,848). One method utilizing fluorescence quenching to analyze Polymerase Chain Reaction (PCR) amplified nucleic acid in a closed tube format is commercially available from Applied Biosystems. The TaqMan™ assay utilizes a nucleic acid hybridization probe, which is labeled with a fluorescent reporter and a quencher moiety in a configuration, which results in quenching of fluorescence in the intact probe. During the PCR amplification, the probe sequence specifically hybridizes to the amplified nucleic acid. When hybridized, the exonuclease activity of the Taq polymerase degrades the probe thereby eliminating the intramolecular quenching maintained by the intact probe. Because the probe is designed to hybridize specifically to the amplified nucleic acid, the increase in fluorescence intensity of the sample, caused by enzymatic degradation of the probe, can be correlated with the activity of the amplification process. Nonetheless, this method preferably requires that each of the fluorophore and quencher moieties be located on the 3′ and 5′ termini of the probe so that the optimal signal to noise ratio is achieved (See: Nazarenko et al., Nucl. Acids Res. 25: 2516-2521 (1997) at p. 2516, col. 2, lns. 27-35). However, this orientation necessarily results in less than optimal fluorescence quenching because the fluorophore and quencher moieties are separated in space and the transfer of energy is most efficient when they are close. Consequently, the background emission from non-hybridized probe can be quite high in the TaqMan™ assay (See: Nazarenko et al., Nucl. Acids Res. 25: at p. 2516, col. 2, lns. 36-40).

The nucleic acid Molecular Beacon is another construct which utilizes fluorescence quenching by the FRET phenomenon to detect target nucleic acid sequences (See: Tyagi et al. Nature Biotechnology, 14: 303308 (1996). A nucleic acid Molecular Beacon comprises a probing sequence embedded within two complementary arm sequences (See: Tyagi et al, Nature Biotechnology, 14: at p. 303, col. 1, lns. 2230). To each termini of the probing sequence is attached one of either a fluorophore or quencher moiety. In the absence of the nucleic acid target, the arm sequences anneal to each other to thereby form a loop and hairpin stem structure which brings the fluorophore and quencher together (See: Tyagi et al., Nature Biotechnology, 14: at p. 304, col. 2, lns. 14-25). When contacted with target nucleic acid, the complementary probing sequence and target sequence will hybridize. Because the hairpin stem cannot coexist with the rigid double helix that is formed upon hybridization, the resulting conformational change forces the arm sequences apart and causes the fluorophore and quencher to be separated (See: Tyagi et al. Nature Biotechnology, 14: at p. 303, col. 2, lns. 1-17). When the fluorophore and quencher are separated, energy of the donor fluorophore does not transfer to the acceptor moiety and the fluorescent signal is then detectable. Since non-hybridized “Molecular Beacons” are non-fluorescent, it is not necessary that any excess probe be removed from an assay. Consequently, Tyagi et al. state that Molecular Beacons can be used for the detection of target nucleic acids in a homogeneous assay and in living cells. (See: Tyagi et al., Nature Biotechnology, 14: at p. 303, col. 2; lns. 15-77). The arm sequences of the disclosed nucleic acid Molecular Beacon constructs are unrelated to the probing sequence (See: Tyagi et al., Nature Biotechnology, 14: at p. 303, col. 1; ln. 30). Because the Tyagi et al. Molecular Beacons comprise nucleic acid molecules, proper stem formation and stability is dependent upon the length of the stem, the GC content of the arm sequences, the concentration of salt in which it is dissolved and the presence or absence of magnesium in which the probe is dissolved (See: Tyagi et al., Nature Biotechnology, 14: at p. 305, col. 1; lns. 1-16). Furthermore, the Tyagi et al. nucleic acid Molecular Beacons are susceptible to degradation by endonucleases and exonucleases. Upon probe degradation, background fluorescent signal will increase since the donor and acceptor moieties are no longer held in close proximity. Therefore, assays utilizing enzymes known to have nuclease activity, will exhibit a continuous increase in background fluorescence as the nucleic acid Molecular Beacon is degraded (See: FIG. 7 in Tyagi et al: the data associated with (0) and (z) demonstrates that the fluorescent background, presumably caused by probe degradation, increases with each amplification cycle). Additionally, Molecular Beacons will also, at least partially, be degraded in living cells because cells contain active nuclease activity.

The constructs described by Tyagi et al. are more broadly described in WO95/13399 (hereinafter referred to as “Tyagi2 et al.” except that Tyagi2 et al. also discloses that the nucleic acid Molecular Beacon may also be bimolecular wherein they define bimolecular as being unitary probes of the invention comprising two molecules (e.g. oligonucleotides) wherein half, or roughly half, of the target complement sequence, one member of the affinity pair and one member of the label pair are present in each molecule (See: Tyagi2 et al., p. 8, ln. 25 to p. 9, ln. 3). However, Tyagi2 et al. specifically states that in designing a unitary probe for use in a PCR reaction, one would naturally choose a target complement sequence that is not complementary to one of the PCR primers (See: Tyagi2 et al., p. 41, ln. 27). Assays of the invention include real-time and end point detection of specific single-stranded or double stranded products of nucleic acid synthesis reactions, provided however that if unitary probes will be subjected to melting or other denaturation, the probes must be unimolecular (See: Tyagi2 et al., p. 37, lns. 1-9). Furthermore, Tyagi2 et al. stipulate that although the unitary probes of the invention may be used with amplification or other nucleic acid synthesis reactions, bimolecular probes (as defined in Tyagi2 et al.) are not suitable for use in any reaction (e.g. PCR) wherein the affinity pair would be separated in a target-independent manner (See: Tyagi2 et al., p. 13, lns. 9-12).

The FRET phenomenon can also be used shift the emission from the fluorescent donor to the fluorescent acceptor resulting in a wavelength shift of the emission (“color shift”). The so-called Hybridization Probes commercialized by Roche Molecular Systems for use with their LightCycler is an example (U.S. Pat. No. 6,174,670). Hybridization Probes comprise two DNA probes hybridizing to adjacent target sequences, where one DNA probe is labeled with a fluorephore (donor) and the other DNA probe is labeled with an other fluorophore (acceptor), such that simultaneous hybridization of the two probes facilitate FRET, such that the emission shifts from the donor fluorophore to the acceptor fluorophore upon excitation of the donor fluorophore. The use of two probes also provide additional specificity as the detectable signal is dependent on the specific hybridization of two DNA probes, however, like the other DNA-based beacon molecules, the hybridization probes are subject to degradation by the endonuclease activity of the Taq DNA polymerase (Wilhelm et al., Biotechniques 30:1052-1062 (2001)).

In order to circumvent the problems related to degradation various PNA probe beacon constructs have been described, and in addition to the high biostability of PNA probes, these probes also take advantage of the improved hybridization characteristics of PNA, such as more rapid kinetics and better discrimination of single base differences. Initially, PNA-based molecular beacons mimicking the molecular beacons by Tyagi et al were described in U.S. Pat. No. 6,355,421, but later a more simple construct without the “arms”—PNA Linear Beacons”—were invented hereby offering a more simple construct facilitating both the probe design and assay development (EP1027372). Yet another self-indicating PNA construct is described in WIPO patent application W097/45539, where the reporter must interact with the nucleic acid to produce signal. Fluorescence quenching by the FRET phenomenon as applied to PNA probes is thus confined to the preparation of simple constructs relying on the specificity of a single PNA probe.

Despite the high specificity of PNA probes, it is well known that a single target sequence does not always provide sufficient specificity or the reliability of an assay may be improved via the added specificity of an additional target sequence. The use of PNA probe mixtures have previously been described as a way to target two adjacent target sequences (US2002058278), however, that concept required each of the two PNA probes hybridizing the adjacent target sequences to be extended with an arm segment capable of forming a triplex with a third labeled PNA probe, such that a total of three probes were required. The use of FRET was neither discussed nor proposed as a way to eliminate the need for “arms” and to avoid the complexity related to a third PNA probe. In fact, the use of a fourth probe or antibody was proposed.

The use of self-reporting probe constructs is primarily described within the context of PCR or other target amplification technologies (U.S. Pat. No. 6,174,670), but also other applications where self-reporting constructs are used to detect the target sequence of interest, such as arrays are being discussed.

It would be desirable to have a system for detecting a target sequence that employs multiple probes and uses fluorescence quenching to detect the target sequence. It would be further desirable to have PNA probes in which at least one of the probes is labeled with a fluorophore and at least one of the other probes is labeled with a quencher. Especially desirable would be to have PNA probe pairs in which hybridization to the target sequence occurs essentially simultaneously and quenches the signal from the probe labeled with the fluorophore.

### SUMMARY OF THE INVENTION

This invention is directed to systems, methods, kits and compositions pertaining to analysis of nucleic acid targets by measuring fluorescence quenching from cohybridization of two PNA probes to adjacent target sequences in hybridization assays.

It has been surprisingly observed that fluorescence quenching by binding of two PNA probes to adjacent target sequences can be correlated to the presence, amount or type of target nucleic acid in a sample. The presence, amount and/or type of target sequence is therefore negatively correlated with the fluorescence signal as illustrated in Table 2. The decrease in observed fluorescence is the result of quenching. One PNA probe is labeled with a fluorophore and one PNA probe is labeled with a quencher oriented such that the fluorophore and the quencher are within quenching proximity when both PNA probes are hybridized to a target. The measurable quenching of fluorescence can be used to detect, identify and/or quantitate the target sequence in a sample.

In one aspect, the invention features a system for detecting a target sequence. Such a system would preferably comprise a pair of PNA probes (PNA probes A and B), wherein probe A is comprised of a nucleotide sequence substantially complementary to a nucleic acid target and is labeled with a fluorophore, Probe B is comprised of a nucleotide sequence substantially complementary to a nucleic acid target adjacent to the target region of PNA probe A and is labeled with a quencher. Preferably, the PNA probes having similar hybridization characteristics such that they hybridize to a similar extent, and at the same time under suitable hybridization conditions. Also preferably, the system includes a detector capable of measuring the change in fluorescence of probe A when probes A and B are hybridized to a target within quenching distance. A preferred detector is capable of outputting the reduction in fluorescence as a signal to a user of the system in real-time or in a computer storable format.

As will be apparent from the discussion and examples that follow, sometimes the pair of PNA probes will be referred to as “PNA Kissing Probes” or a like phrase to denote the close hybridization association between a fluorophore labeled probe and a quencher labeled probe. In use, preferred PNA probes upon hybridization to a desired target will be separated by less than about 5 base pairs, preferably less than about 3 base pairs. For most embodiments, the PNA Kissing probes will be separated by less than about 1 base pair, usually 0 base pairs (adjacent) upon hybridization to the template so as to minimize the distance between the fluorophore and quencher labels of the probes.

PNA probe pairs are envisioned where one probe is labeled with a fluorophore, and the other probe is labeled with a quencher such that when both probes are brought into close proximity though hybridization to a target, there is a measurable decrease in fluorescence of the fluorophore. The respective fluorophore and quencher labels are attached to the probes such that upon hybridization the labels are oriented to allow some level of quenching to be measured. The distance between and orientation of the respective fluorophore and quencher labels are described as “within quenching distance” when some measurable level of quenching can be observed. The fluorophore or quencher labels may be attached to the probes in any configuration such that the labels are with quenching distance when the probes are hybridized to a mutually complementary target. Potential configurations of the labels include N-terminally, C-terminally, or internally. Labels may also be bound to the main sequence of the probes by any of a variety of linkers. In preferred embodiments, the PNA probe pair is designed such that one probe is labeled N-terminally and the other is labeled C-terminally, and the probe which is labeled N-terminally hybridizes upstream (5 prime) to the probe which is labeled C-terminally.

The invention is not limited by the mechanism of fluorescent quenching, and is meant to include fluorescent quenching which occurs by FRET and non-FRET mechanisms.

The present invention also provides useful methods for the analysis of a target sequence in a sample. In one embodiment, the method includes at least one and preferably all of the following steps:

a. contacting the sample with a mixture comprising:

A mixture of two PNA probes (PNA probe A and PNA probe B), wherein:

i) PNA probe A is comprised of a nucleotide sequence, which cohybridizes to a target region of DNA or RNA and is labeled with a fluorophore at the end which, upon hybridization is closest to the adjacent target region for PNA probe B; and
ii) PNA probe B is comprised of a nucleotide sequence, which hybridizes to a target region of DNA or RNA adjacent to the target region of PNA probe A and is labeled with a quencher at the end which, upon hybridization is closest to the adjacent target region for PNA probe A.
b. measuring the fluorescence from the hybridization of PNA probe A and PNA probe B to the target sequence, under suitable hybridization conditions, wherein the presence or amount of target sequence present in the sample can be negatively correlated with the fluorescence of the fluorophore on PNA probe A.

Further provided is an array that includes two or more PNA Probe A immobilized to a surface support, wherein simultaneous hybridization of pairs of PNA Probe A and PNA Probe B to adjacent target sequences at predetermined locations are suitable for detecting, identifying or quantitating a two or more target sequences present in a sample.

The invention also features a kit that includes at least one of the pairs of PNA probes disclosed herein.

In another aspect, the invention provides a method for the analysis of a target sequence in a sample (sometimes called a “first” sample). In one embodiment, the method provides at least one and preferably all of the following steps: contacting the first sample with a mixture that includes a mixture of two probes (probe A and probe B), in which:

i) Probe A includes of a nucleotide sequence, directed to region of the target of DNA or RNA adjacent to the target region of Probe A and is labeled with a quencher which, upon hybridization is closest to the adjacent target region for Probe A. Preferably, the method further includes a step b. involving measuring fluorescence following cohybridization of Probe A and Probe B to the target sequence, under suitable hybridization conditions, in which presence or amount of target sequence present in the first sample is negatively correlated with the fluorescence of the fluorophore on Probe A.

In another aspect, the invention provides an array that in a preferred embodiment includes a probe A immobilized to a surface support in which one or more pairs of Probe A and Probe B cohybridise to adjacent target sequences. Typically, such cohybridisation can be used to detect the target sequences in line with the invention. Also typically, the cohybridisation to the target is to one or more predetermined locations suitable for analysis of two or more target sequences in a sample.

The present invention provides advantages. For instance, fluorescence quenching occurs upon hybridization of the PNA Kissing Probes to their target sequences in contrast to other self-reporting PNA constructs previously described where fluorescence quenching occurs in the non-hybridized state. This is a fundamental difference that offers significant performance advantages, such as higher specificity and/or sensitivity as will be discussed below leading to more reliable and robust assay formats.

The invention provides additional advantages.

For instance, the simultaneous hybridization of the two PNA probes provides increased specificity as compared to conventional hybridization assay using one PNA probe since the measurement is dependent on the hybridization to two adjacent target regions instead of one target region. When compared to a control target the base sequence of a sample target may be surmised. For example, a sample target which contains a base insertion between the binding regions of Probes A and B will likely demonstrate less quenching than a control target where the binding regions are immediately adjacent. Optimal performance is ensured by adjusting the nucleobase sequences, such that the two PNA probes have similar hybridization characteristics.

Other advantages are provided by the invention. For instance, in the absence of target, or in the absence of a continuous target there is no fluorescence quenching. The baseline signal is therefore equivalent to the maximum fluorescence from the fluorophore and therefore easily measurable. Fluorescence quenching due to the presence of target is therefore readily detectable as a decrease from the maximum signal. In contrast, when using the minimum fluorescence as baseline as being the case for all other DNA and PNA-based self-reporting constructs previously described, the fluorescence due to the presence of target has to exceed the measurable limit and/or the baseline before being measurable. The negative correlation between fluorescence and the amount of target for PNA Kissing Probes therefore supports a lower detection limit (higher sensitivity) as compared to the positive correlation between fluorescence and the amount of target for all other self-reporting DNA or PNA probe constructs.

The invention is particularly well-suited for real-time PCR applications where the formation of target can be specifically detected, identified and/or quantitated. The negative correlation between fluorescence and target amount circumvents some of the current difficulties with real-time PCR applications, such as the inherent fluorescence of beacons in their non-hybridized stage (background) and the establishment of a reliable baseline. More importantly, the formation of amplicon is from the beginning within the measurable range as discussed above and is therefore not dependent on the formation of sufficient amplicon to generate a signal above the baseline and/or within the measurable range. Thus, this preferred embodiment provides both improved specificity due the use of two PNA probes and improved sensitivity due to a lower detection limit resulting from the negative correlation between fluorescence and amount of target.

PNA probes are the preferred constructs for this invention due to their improved hybridization characteristics and high biostability, but it is understood that other probe constructs, such as LNA, or regular DNA probes and modifications thereof are within the embodiment of the invention. In respect to probe design, LNA probes offers advantages as their hybridization characteristics may be adjusted by the number and positions of the LNA nucleotides within the probe. Also the combination of different probe constructs are within the embodiment of the invention and illustrated in Example 2, where Probe A is a DNA probe and Probe B is a PNA probe.

### DETAILED DESCRIPTION OF THE INVENTION

As discussed, the invention features a system, methods, kits and compositions suitable for the analysis of nucleic acid target sequences using PNA Kissing Probes.

As discussed, and in one embodiment, the invention provides for a system for analysis a target sequence. In one embodiment, the system includes:

a) a pair of PNA probes (PNA probe A and PNA probe B), wherein PNA probe A is comprised of a nucleotide sequence complementary to a target region of DNA or RNA and is labeled with a fluorophore at the end which, upon cohybridization is closest to the adjacent target region for PNA probe B;
PNA probe B is comprised of a nucleotide sequence complementary to a target region of DNA or RNA adjacent to the target region of PNA probe A and is labeled with quencher at the end which, upon hybridization is closest to the adjacent target region for PNA probe A, said fluorophore and quencher being the donor moiety and acceptor moiety, respectively, for fluorescence quenching, and said PNA probes having similar hybridization characteristics such that they cohybridize under suitable hybridization conditions; and
b) a detector adapted to measure quenching of PNA probe A upon hybridization of PNA probe B to the target region.

By “close”, “closest” and like words or phrases when used in reference to position between the Probe A fluorophore and quencher of Probe B is meant spacing between same of less than about 10 nucleotides apart, preferably less than 8 nucleotides apart, more preferably about 0 to about 5 nucleotides apart. _{[HS1]}

By a “detector” is meant an automated, semi-automated or in some cases manual device that is intended to receive signal and register output to a user of the invention either directly or indirectly (e.g., via computer assistance). Examples of suitable detectors include, but are not limited to, devices that have capacity to receive and register fluorescence signal such as a spectrofluorometric, real-time PCR instrument, flow cytometer, fluorescence microscope, array scanner and like devices.

An “end” of a DNA or RNA molecule as disclosed herein means the 5′ or 3′ end unless specified otherwise. Reference herein to “internal” probe nucleobases (as in labeling a probe internally, for instance), means not at the end of a probe but therebetween, ie., one or more nucleobases between the ends of that probe. _{[HS2]}

Nearly any suitable probe sequence can be used to practice the invention. Useful PNA probe sequences will be less than about 30 PNA subunits, preferably less then about 20 PNA subunits, preferably between from about 8 to 18 PNA subunits with between from about 11 to about 16 PNA subunits being preferred for many applications.

By the phrase ‘cohybridize’ is meant that PNA probe A and PNA probe B hybridize during the same assay step using the same hybridization conditions. Specifically provided is a method of making the probes disclosed herein which methods include in one embodiment, the further step of comparing nucleobase sequences of DNA or RNA. Typically, such a method further includes the step of selecting two PNA nucleobase sequence complementary to adjacent regions of the target sequence, where both target regions include at least one nucleobase differing from the corresponding non-target seunces, eg., less than about 10 of such nucleobases such as one, two or three of same. Also typically, the method includes the step of adjusting (modifying) the two PNA nucleobase sequences as needed to ensure similar hybridization characteristics under suitable hybridization conditions.

By the phrase “similar hybridization characteristics” is meant that the subject sequences of interest (eg., PNA probes) hybridize under the same hybridization characteristics. Examples of suitable hybridization conditions (sometimes referred to as “high”, “medium” or “low” stringency conditions, for example) can be found in Sambrook et al. in Molecular Cloning: A Laboratory Manual (2d ed. 1989); and Ausubel et al., Current Protocols in Molecular Biology, John Wiley & Sons, New York, 1989. An illustrative hybridization condition (“high stringency”) can be found in Example 3.

In another embodiment, the method of making the probes disclosed herein, includes the step of comparing nucleobase sequences of DNA or RNA. Typically, the method further includes selecting two PNA nucleobase sequences that are complementary to adjacent regions of the target sequence, where at least one of the target regions includes at least one nucleobase differing from the corresponding non-target sequences. Also typically, the method includes adjusting the two PNA nucleobase sequences to ensure similar hybridization characteristics under suitable hybridization conditions.

Suitable probes for use with the invention feature, upon hybridization, a separation between the two PNA probes of a distance of less then about ten nucleotide base pairs, preferably about one to five nucleotide bases as long usually as energy transfer occurs. More preferably, there is no separation between the two PNA probes ie., the probes are adjacent to one another.

As will be appreciated, the invention is not tied to the use of any particular fluorophore and many known in the field will be suitable for a variety of application. In one embodiment, an acceptable fluorophore for use with the invention is selected from the group consisting of 5(6)-carboxyfluorescein, 5-(2′-aminoethyl)-aminonaphthalene-1-sulfonic acid (EDANS), bodipy, rhodamine, Cy2, Cy3, Cy 3.5, Cy5, Cy5.5 and texas red. Appropriate quencing entities are also known and include, but are not limited to, 4-((4-(dimethylamino)phenyl)azo)benzoic acid (dabcyl).

As also discussed, the invention and particularly the system or pair of PNA probes disclosed herein can include one or more spacer moieties linked to one or both of the donor and acceptor moieties to the end of the PNA probes to which it/they is/are linked. A typical spacer moiety includes one or more linked amino acid moieties.

The present invention is flexible and can be used in one or a combination of suitable formats. For instance, the system or pair of PNA probes of the invention can be to a support, usually a solid support.

A variety of probes and probe pairs can be used with the invention provided intended detection results are achieved. Also envisioned are the complementary sequences thereof as well as variants of the probe sequences and complementary sequences. By “complementary” or “complementary sequence” when referring to a probe or pair thereof is meant a nucleic acid or PNA oligomer designed to hybridize with exact complementarity to the subject probe. A “variant” of the probe sequence includes nucleobase derivatives eg., methylcytosine, 2-aminopurine, 2-amino-6-chloropurine, 2,6-diaminopurine, hypoxanthine, pseudoisocytosine, 2-thiouracil, 2-thiothymidine uracil, and the like; as well as nucleoside deletions (sequential or non-contiguous) or substitutions (eg., T=>U) that do not impact hybridization to the corresponding complementary sequence by more than about 2° C.

As discussed, the invention also provides for a method for detecting, identifying or quantitating a target sequence of interest in a sample. In one embodiment, the method is used to detect target sequence in a closed tube (homogeneous) assay. In another embodiment, the method is used to detect a nucleic acid comprising a target sequence wherein said nucleic acid has been synthesized or amplified in a reaction occurring in the closed tube (homogeneous) assay. Typical nucleic acid synthesis or nucleic acid amplification reactions for use with the method are selected from the group consisting of: Polymerase Chain Reaction (PCR), Ligase Chain Reaction (LCR), Strand Displacement Amplification (SDA), Transcription-Mediated Amplification (TMA), Rolling Circle Amplification (RCA) and Q beta replicase. In one embodiment, the PCR reaction is an asymmetric PCR reaction.

The invention provides additional methods for analysis of a target sequence of interest in a sample. In one illustration of the method, it is used to detect a target sequence in a cell or tissue. Such a cell or tissue may or may not be living.

In one embodiment of the foregoing methods, the sample to be analyzed is contacted with said pairs of PNA probes and one or more blocking probes.

Methods of the invention have a wide spectrum of useful applications including use to detect, identify, or quantitate the presence or amount of an organism or virus in the sample. Alternatively, or in addition, the method is used to detect, identify, or quantitate the presence or amount of one or more species of an organism in the sample. For instance, the method can be used to determine the effect of antimicrobial agents on the growth of one or more microorganisms in the sample. Also, such methods can be used to determine the presence or amount of a taxonomic group of organisms in the sample. The methods also find use to diagnose a condition of medical interest.

The invention is compatible with a wide variety of target sequences including those immobilized to a surface. Alternatively, or in addition, the PNA probe is immobilized to a surface.

A particular array in accord with this invention is suitable for regeneration by treatment with one or more regeneration catalyst selected from the group consisting of heat, nuclease enzyme and chemical denaturant such as aqueous solutions containing formamide, urea and or sodium hydroxide.

As further disclosed herein, the invention provides kits for performing an assay or method that helps detect the presence, absence or amount of target sequence. In one embodiment, one of more of the polymers of the kit have a probing nucleobase sequence of less than about 20 subunits, preferably about 11-16 subunits in length. Alternatively, or in addition, two or more polymers in the kit are labeled with independently detectable moieties. Typical of such moieties are those used to independently detect, identify or quantitate at least two different target sequences which may be present in the same sample.

The kits can be used to detect organisms in food, beverages, water, pharmaceutical products, personal care products, dairy products or environmental samples. Additional kits of the invention can be used to test raw materials, products or processes, clinical samples such as clinical specimens or equipment, fixtures and products used to treat humans or animals.

Additional kits can be used to detect a target sequence which is specific for a genetically based disease or is specific for a predisposition to a genetically based disease. In one embodiment, the kit is used to detect a target sequence associated with a disease selected from the group consisting of 5-Thalassemia, sickle cell anemia, Factor-V

Leiden, cystic fibrosis and cancer related targets such as p53, pIO, BRC-1 and BRC-2.

Further kits of the invention can be used to detect a target sequence in a forensic technique such as prenatal screening, paternity testing, identity confirmation or crime investigation.

The invention is flexible and can be used in one or a combination of suitable probe formats. In one embodiment, the quencher-labeled PNA probe is a PNA linear beacon with a fluorophore at the opposite end, which differs from the fluorophore on the fluorophore-labeled PNA probe. Presence of the fluorescent signal will then depend on whether just the fluorophore-labeled PNA probe is bound or whether both fluorophore-labeled and the linear PNA beacon are bound. For example, in an embodiment in which the linear PNA beacon is labeled with fluorescein (green) and a quencher at opposite ends and targeting a unique target sequence whereas the fluorophore-labeled PNA probe is labeled with rhodamine (red) and targeting an adjacent universal sequence. The signal will then be red for target s that do not have the unique sequence whereas the signal for target sequences with the unique target sequence will be green, only, as the red signal will be quenched. This approach is particularly well-suited for design of dual-color assays with inherent negative controls.

### Fluorescence Quenching:

Hybrid formation of PNA Kissing Probes with adjacent target sequences can be monitored by measuring the decrease in the fluorescence of the fluorophore linked to one of the two PNA probes as compared with the fluorescence in the absence of target sequence. We refer to this phenomenon as fluorescence quenching. The quenching is due to an increase in the efficiency of energy transfer between the fluorophore (donor moiety) and quencher (acceptor moiety) caused by cohybridization of a pair of PNA Probes to adjacent target sequences. Measuring the fluorescence quenching is used to detect, identify or quantitate hybridization of the PNA Kissing Probes to the adjacent target sequences.

### Spacer/Linker Moieties:

Generally, spacers are used to minimize the adverse effects that bulky labeling reagents might have on hybridization properties of probes. Linkers typically induce flexibility and randomness into the probe or otherwise link two or more nucleobase sequences of a probe. Preferred spacer/linker moieties for the nucleobase polymers of this invention consist of one or more aminoalkyl carboxylic acids (e.g. aminocaproic acid) the side chain of an amino acid (e.g. the side chain of lysine or ornithine) natural amino acids (e.g. glycine), aminooxyalkylacids (e.g. 8-amino-3,6-dioxaoctanoic acid), alkyl diacids (e.g. succinic acid), alkyloxy diacids (e.g. diglycolic acid) or alkyldiamines (e.g. 1,8-diamino-3,6-dioxaoctane).

Spacer/linker moieties may also incidentally or intentionally be constructed to improve the water solubility of the probe (For example see: Gildea et al., Tett. Lett. 39: 7255-7258 (1998)).

Preferably, a spacer/linker moiety comprises one or more linked compounds having the formula: —Y—(Om-(CW2)n)O-Z-. The group Y is selected from the group consisting of: a single bond, —(CW2)p-, —C(O)(CW)p-, —C(S)(CW2) p- and —S(O)(CW)P. The group Z has the formula NH, NR2, S or O. Each W is independently H, R2, oR2, F, Cl, Br or 1; wherein, each W is independently selected from the group consisting of: —CX3, —CX2CX3, —CX2CX2CX3, —CX2CX(CX3)2, and —C(CX3)3. Each X is independently H, F, Cl, Br or I. Each m is independently 0 or 1. Each n, o and p are independently integers from 0 to 10.

### Hybridization Conditions/Stringency:

Those of ordinary skill in the art of nucleic acid hybridization will recognize that factors commonly used to impose or control stringency of hybridization include formamide concentration (or other chemical denaturant reagent), salt concentration (i.e., ionic strength), hybridization temperature, detergent concentration, pH and the presence or absence of chaotropes. Optimal stringency for a probe/target sequence combination is often found by the well known technique of fixing several of the aforementioned stringency factors and then determining the effect of varying a single stringency factor. The same stringency factors can be modulated to thereby control the stringency of hybridization of a PNA to a nucleic acid, except that the hybridization of a PNA is fairly independent of ionic strength. Optimal stringency for an assay may be experimentally determined by examination of each stringency factor until the desired degree of discrimination is achieved.

### Suitable Hybridization Conditions:

Generally, the more closely related the non-target sequences are to the target sequence, the more carefully stringency must be controlled. Blocking probes may also be used as a means to improve discrimination beyond the limits possible by mere optimization of stringency factors. Suitable hybridization conditions will thus comprise conditions under which the desired degree of discrimination is achieved such that an assay generates an accurate (within the tolerance desired for the assay) and reproducible result.

Aided by no more than routine experimentation and the disclosure provided herein, those of skill in the art will easily be able to determine suitable hybridization conditions for performing assays utilizing the methods and compositions described herein. Suitable in-situ hybridization or PCR conditions comprise conditions suitable for performing an in-situ hybridization or PCR procedure. Thus, suitable in-situ hybridization or PCR conditions will become apparent to those of skill in the art using the disclosure provided herein; with or without additional routine experimentation.

### Hybridization Characteristics:

The hybridization characteristics of a probe are usually described by the melting point (Tm) of the probe-target hybrid. The melting point is therefore an important parameter used to guide the experimentation described above to determine the suitable hybridization conditions. However, when the assay is dependent on simultaneous hybridization of two PNA probes each of these two PNA probes must to be designed with similar hybridization characteristics such that the same hybridization conditions are suitable for both PNA probes. The length of the nucleobase sequence provides a rough assessment of the hybridization characteristics, but can be refined by calculating the Tm using on-line calculators available at www.appliedbiosystems.com. The degree of similarity between the hybridization characteristics of PNA Kissing Probes is dependent on both the stringency of the hybridization conditions and the desired degree of discrimination that needs to be achieved. Aided by no more than routine experimentation and the disclosure provided herein, those of skill in the art will easily be able to determine the degree of similarity required for performing assays utilizing the methods and compositions described herein.

### Blocking Probes:

Blocking probes are nucleic acid or non-nucleic acid probes that can be used to suppress the binding of the probing nucleobase sequence of the probing polymer to a nontarget sequence. Preferred blocking probes are PNA probes (See: Coull et al., U.S. Pat. No. 6,110,676).

Typically blocking probes are closely related to the probing nucleobase sequence and preferably they comprise one or more single point mutations as compared with the probe sought to be detected in the assay. It is believed that blocking probes operate by hybridization to the non-target sequence to thereby form a more thermodynamically stable complex than is formed by hybridization between the probing nucleobase sequence and the non-target sequence. Formation of the more stable and preferred complex blocks formation of the less stable non-preferred complex between the probing nucleobase sequence and the non-target sequence. Thus, blocking probes can be used with the methods, kits and compositions of this invention to suppress the binding of the nucleic acid or non-nucleic acid probe to a non-target sequence that might be present and interfere with the performance of the assay.

Blocking probes are particularly advantageous in single point mutation discrimination.

### Probing Nucleobase Sequence:

The probing nucleobase sequence of a probe of this invention is the specific sequence recognition portion of the construct. Therefore, the probing nucleobase sequence is a nucleobase sequence designed to hybridize to a specific target to directly or indirectly detect the presence, absence or amount of the target sequence of interest in a sample. Consequently, with due consideration to the requirements of a probe for the assay format chosen, the length and sequence composition of the probing nucleobase sequence of the probe will generally be chosen such that a stable complex is formed with the target sequence under suitable hybridization conditions.

This invention contemplates that variations in these identified probing nucleobase sequences shall also provide suitable probes. Variation of the probing nucleobase sequences within the parameters described herein are considered to be an embodiment of this invention. Common variations include, deletions, insertions and frame shifts. Additionally, a shorter probing nucleobase sequence can be generated by truncation of the above identified sequence.

A probe of this invention will generally have a probing nucleobase sequence that is exactly complementary to the target sequence. Alternatively, a substantially complementary probing nucleobase sequence might be used since it has been demonstrated that greater sequence discrimination can be obtained when utilizing probes wherein there exists one or more point mutations (base mismatch) between the probe and the target sequence (See: Guo et al., Nature Biotechnology 15: 331-335 (1997)). Consequently, the probing nucleobase sequence may be only 90% homologous to the probing nucleobase sequences identified above. Substantially complementary probing nucleobase sequence within the parameters described above are considered to be an embodiment of this invention.

Complements of the probing nucleobase sequence are considered to be an embodiment of this invention, since it is possible to generate a suitable probe if the target sequence to be detected has been amplified or copied to thereby generate the complement to the identified target sequence.

Detection, Identification and/or Quantitation

Detection is meant analysis for the presence or absence of the target sequence optionally present in the sample. Identification is meant establishment of the identity of the target sequence. By quantitation is meant measuring the amount of target sequence in a sample. Some assay formats provide simultaneous detection, identification and enumeration (for example see Stender, H. et al., J. Microbiol. Methods. 45:31-39 (2001), others provide detection and identification (foe example see Stender, H. et al., Int. J. Tuberc. Lung Dis. 3:830-837 (1999) and yet other assay formats only provide identification (for example see Oliveira, K et al. J. Clin. Microbiol. 40:247-251 (2002)).

### II. PREFERRED EMBODIMENTS OF THE INVENTION

### a PNA Kissing Probes

This invention pertains to methods, kits and compositions pertaining to pairs of PNA Probes, so-called PNA Kissing Probes. Pairs of PNA Probes efficiently transfer energy between the fluorophore and the quencher linked to adjoining ends of two PNA probes hybridized to adjacent target sequences leading to fluorescence quenching. Quenching of the fluorescence signal of the fluorophore can be used to monitor or quantitate the occurrence of the hybridization event.

Generally, PNA Kissing Probes is a pair of PNA probe suitable for detecting, identifying or quantitating a target sequence. At a minimum, one PNA Probe comprises a probing nucleobase sequence and one linked fluorophore and the other PNA Probe comprises a probing nucleobase sequence and one linked quencher. The fluorophore and quencher are linked to the adjoining ends of the two PNA Probes when hybridized to adjacent target sequences. The fluorophore and quencher are located at opposite termini of the two PNA Probe but either orientation of the labels is acceptable. The PNA Kissing Probes are further characterized in that detectable fluorescence quenching occurs upon hybridization to the adjacent target sequences under suitable hybridization conditions.

Preferably, PNA Kissing Probes are assembled by stepwise condensation of suitably protected moieties. Consequently, the PNA polymer is preferably continuous from the amino to the carboxyl terminus. In the most preferred configuration, PNA Kissing Probes are continuous from the N-terminus to the C-terminus and the probing nucleobase sequence is oriented toward the C-terminus of the polymer.

The PNA probes of this invention will comprise at least the probing nucleobase sequence (as previously described herein) and a fluorophore or a quencher, but may comprise additional moieties. Non-limiting examples of additional moieties include linkers, spacers, natural or non-natural amino acids, or other subunits of PNA, LNA, DNA or RNA.

In preferred embodiments, PNA Kissing Probes comprising the selected probing nucleobase sequence described herein are particularly useful in all kinds of homogeneous assays such as in real-time PCR or useful with self-indicating devices (e.g. lateral flow assay) or self-indicating arrays.

This embodiment of PNA Kissing Probes is unique even in light of the Hybridization Probes, PNA Molecular Beacons and other self-reporting DNA or PNA constructs as will be discussed below.

### b Unique Features of PNA Kissing Probes

There are many biological, structural, and physico-chemical differences between prior art DNA and PNA constructs and the PNA Kissing Probes of this invention:

DNA based constructs, such Molecular Beacons and Hybridization Probes, comprise a polynucleotide backbone whereas the PNA Kissing Probes of this invention comprise a probing nucleobase sequence which is not a polynucleotide. Thus, PNA Kissing Probes which comprise PNA subunits exhibit all of the favorable properties of PNA such as resistance to nuclease degradation, salt independent sequence hybridization to complementary nucleic acids and rapid hybridization kinetics.

Several of the DNA and PNA constructs, such Molecular Beacons and PNA Molecular Beacons, have “arm” segments which are embedded within the probing nucleobase sequence hereby increasing the complexity of the assay design.

All PNA constructs are based on a single PNA probe, whereas PNA Kissing Probes offers added specificity due to the use of two probes.

All other DNA and PNA construct, except Hybridization Probes, are designed such that FRET occur in the non-hybridized stage, whereas PNA Kissing Probes function via FRET occurring in the hybridized stage.

Hybridization Probes and PNA Kissing Probes differs fundamentally even if Hybridization Probes where made of PNA. Hybridization Probes provide positive correlation between the signal and the amount of target whereas PNA Kissing Probes provide negative correlation between signal and the amount of target. A difference that is due to the fact that the measurable signal from Hybridization Probes comes from the emission of the acceptor fluorophore whereas the measurable signal from the PNA Kissing Probes comes from the emission of the donor fluorophore. Because of this difference, it is the donor moiety that is labeled with independently detectable fluorephore when two of more sets of PNA Kissing Probes are applied for multiplex assays as described in more details below, whereas in case of Hybridization Probes it is the acceptor fluorophores that differ. A difference that offers greater flexibility in the selection of fluorophore. PNA Kissing Probes are therefore not dependent on the emission from the acceptor moiety which allow the use of standard filter sets for the fluorophores whereas the Hybridization Probes need a combination of the excitation filter for the donor fluorophore and emission filter for the acceptor fluorophore. The use of Hybridization Probes is therefore limited to selected donor-acceptor paris and instrumentation, such as the LightCycler, and not directly applicable for conventional fluorophore and instrumentation.

c. Sets of PNA Kissing Probes

In another embodiment, this invention is directed to sets of PNA Kissing Probes suitable for detecting or identifying the presence, absence or amount of two or more different target sequences, which might be present in a sample. The characteristics of sets of PNA Kissing Probes suitable for the detection, identification or quantitation of target sequences have been previously described herein.

d. Immobilization of a PNA Kissing Probes to a Surface

One or more fluorophore-labeled PNA probes of one or more sets of PNA Kissing Probes may optionally be immobilized to a surface. In one embodiment, the probe can be immobilized to the surface using the well-known process of UV-crosslinking. Alternatively, the PNA oligomer is synthesized on the surface in a manner suitable for deprotection but not cleavage from the synthesis support.

Preferably, the probe is covalently linked to a surface by the reaction of a suitable functional groups on the probe and support. Functional groups such as amino groups, carboxylic acids and thiols can be incorporated in a PNA Probe by extension of one of the termini with suitable protected moieties (e.g. lysine, glutamic acid and cystine). When extending the terminus, one functional group of a branched amino acid such as lysine can be used to incorporate the donor or acceptor label at the appropriate position in the polymer (See: Section entitled “PNA Labeling”) while the other functional group of the branch is used to optionally further extend the polymer and immobilize it to a surface.

Methods for the attachment of probes to surfaces generally involve the reaction of a nucleophilic group, (e.g. an amine or thiol) of the probe to be immobilized, with an electrophilic group on the support to be modified. Alternatively, the nucleophile can be present on the support and the electrophile (e.g. activated carboxylic acid) present on the PNA Molecular Beacon. Because native PNA possesses an amino terminus, a PNA will not necessarily require modification to thereby immobilize it to a surface (See: Lester et al., Poster entitled “PNA Array Technology”).

Conditions suitable for the immobilization of a PNA to a surface will generally be similar to those conditions suitable for the labeling of a PNA (See: subheading “PNA Labeling”). The immobilization reaction is essentially the equivalent of labeling the PNA whereby the label is substituted with the surface to which the PNA probe is to be covalently immobilized.

Numerous types of surfaces derivatized with amino groups, carboxylic acid groups, isocyantes, isothiocyanates and malimide groups are commercially available. Non-limiting examples of suitable surfaces include membranes, glass, controlled pore glass, polystyrene particles (beads), silica and gold nanoparticles.

When immobilized to a surface, there will be no fluorescence quenching of the immobilized PNA probe. Upon hybridization to a target sequence under suitable hybridization conditions, the location on the surface where the fluorophore-labeled PNA Probe (of known sequence) is attached and simultaneous hybridization of the quencher-labeled PNA probe to the adjacent target sequence will generate fluorescence quenching. Consequently, the intensity of the signal on the surface can be used to detect, identify or quantitate the presence or amount of a target sequence in a sample, which contacts the surface to which the fluorophore-labeled PNA Probe is immobilized. As previously discussed there will be a negative correlation between the signal and the amount of target.

e. Detectable and Independently Detectable Moieties/Multiplex Analysis

In preferred embodiments of this invention, a multiplex hybridization assay is performed. In a multiplex assay, numerous conditions of interest are simultaneously examined. Multiplex analysis relies on the ability to sort sample components or the data associated therewith, during or after the assay is completed. In preferred embodiments of the invention, distinct independently detectable moieties are used to label the different fluorophore-labeled PNA probes of the sets of PNA Kissing Probes. It should be noted that the same quencher can be used for the quencher-labeled PNA probe of the pairs of PNA Kissing Probes. The ability to differentiate between and/or quantitate each of the independently detectable fluorophores provides the means to multiplex a hybridization assay because the data which correlates with the hybridization of each of the distinctly (independently) labeled PNA Kissing Probes to a target sequence can be correlated with the presence, absence or quantity of the target sequence sought to be detected in a sample. Consequently, the multiplex assays of this invention may be used to simultaneously detect the presence, absence or amount of one or more target sequences, which may be present in the same sample in the same assay. Preferably, independently detectable fluorophores will be used in a multiplex assay using two or more sets of PNA Kissing Probes. For example, two sets of PNA Kissing Probes might be used to detect each of two different target sequences wherein a fluorescein (green) labeled probe would be used to detect the first of the two target sequences and a rhodamine or Cy3 (red) labeled probe would be used to detect the second of the two target sequences. Consequently, a red, a green or no signal in the assay would signify the presence of the first, second and first and second target sequences, respectively.

f. Arrays of PNA Kissing Probes

Arrays are surfaces to which two or more probes of interest have been immobilized at predetermined locations. Arrays comprising both nucleic acid and PNA probes have been described in the literature. The probe sequences immobilized to the array are judiciously chosen to interrogate a sample which may contain one or more target sequences of interest. Because the location and sequence of each probe is known, arrays are generally used to simultaneously detect, identify or quantitate the presence or amount of one or more target sequences in the sample. Thus, PNA arrays may be useful in diagnostic applications or in screening compounds for leads, which might exhibit therapeutic utility.

An array using PNA Kissing Probes will comprise the fluorophore-labeled PNA probe on the surface of the array and the quencher-labeled PNA probe applied together with the sample of interest. Since the composition of the fluorophore-labeled PNA probe is known at the location on the surface of the array (because the PNA was synthesized or attached to this position in the array), the composition of target sequence(s) can be directly detected, identified or quantitated by determining the location of decreased signal generated in the array. Because hybridization of the pairs of PNA Kissing Probes to a target sequence is self-indicating, no secondary detection system is needed to analyze the array for hybridization between the PNA Kissing Probes and the target sequence.

PNA arrays should be reusable provided the nucleic acid from one sample can be striped from the array prior to introduction of the second sample. Upon stripping of hybridized target sequences, signal on the array of fluorophore-labeled PNA probe should again reach maximum fluorescence. This provide a good control prior to reusing the array as opposed to array using PNA Molecular Beacons, where the signal upon regeneration is reduced to background and thus indistinguishable from degraded PNA Molecular Beacons. Because PNAs are not degraded by heat or endonuclease and exonuclease activity, arrays of PNA Kissing Probes should be suitable for simple and rapid regeneration by treatment with heat, nucleases or chemical denaturants such as aqueous solutions containing formamide, urea and/or sodium hydroxide.

g. Methods

In yet another embodiment, this invention is directed to a method for the detection, identification and/or quantitation of a target sequence in a sample. The method comprises contacting the sample with PNA Kissing Probes and then detecting, identifying and/or quantitating the fluorescence signal of the fluorophore whereby correlation between detectable signal and hybridization is possible since PNA Kissing Probes are self-indicating. Because PNA Kissing Probes are self-indicating, this method is particularly well suited to analysis performed in a closed tube assay (also referred to in the field as “homogeneous assays”). By closed tube assay we mean that once the component of the assay have been combined, there is no need to open the tube or remove contents of the assay to determine the result. Since the tube need not, and preferably will not, be opened to determine the result, there must be some detectable or measurable change which occurs and which can be observed or quantitated without opening the tube or removing the contents of the assay. Thus, most closed tube assays rely on a change in fluorescence which can be observed with the eye or otherwise be detected and/or quantitated with a fluorescence instrument which uses the tube as the sample holder. Examples of such instruments include the Light Cycler from Idaho Technologies and the Prism 7700 from Perkin Elmer.

Preferred closed tube assays of this invention comprise the detection of nucleic acid target sequences, which have been synthesized or amplified by operation of the assay. Non-limiting examples of preferred nucleic acid synthesis or nucleic acid amplification reactions are Polymerase Chain Reaction (PCR), Ligase Chain Reaction (LCR), Strand Displacement Amplification (SDA), Transcription-Mediated Amplification (TMA), Rolling Circle Amplification (RCA) and Q-beta replicase. The PNA Kissing Probes present in the closed tube assay will generate fluorescence quenching in response to target sequence production from the nucleic acid synthesis or nucleic acid amplification reaction occurring in the closed tube assay. In a most preferred embodiment, the assay is an asymmetric PCR reaction.

Because the method of this invention may be used in a probe-based hybridization assay, this invention will find utility in improving assays used to detect, identify of quantitate the presence or amount of microorganisms in a sample through the detection of target sequences associated with the microorganisms. (See: U.S. Pat. No. 5,641,631, entitled “Method for detecting, identifying and quantitating organisms and viruses” herein incorporated by reference): Similarly, this invention will also find utility in an assay used in the detection, identification or quantitation of one or more species of an organism in a sample (See U.S. Pat. No. 5,288,611, entitled “Method for detecting, identifying and quantitating organisms and viruses” herein incorporated by reference). This invention will also find utility in an assay used to determine the effect of antimicrobial agents on the growth of one or more microorganisms in a sample (See: U.S. Pat. No. 5,612,183, entitled “Method for determining the effect of antimicrobial agents on growth using ribosomal nucleic acid subunit subsequence specific probes” herein incorporated by reference). This invention will also find utility in an assay used to determine the presence or amount of a taxonomic group of organisms in a sample (See: U.S. Pat. No. 5,601,984, entitled “Method for detecting the presence of amount of a taxonomic group of organisms using specific r-RNA subsequences as probes” herein incorporated by reference).

When performing the method of this invention, it may be preferable to use one or more unlabeled or independently detectable probes in the assay to thereby suppress the binding of the PNA Kissing Probes to a non-target sequence. The presence of the “blocking probe(s)” helps to further increase the discrimination of the assay and thereby improve reliability and specificity.

In certain embodiments of this invention, one target sequence is immobilized to a surface by proper treatment of the sample. Immobilization of the nucleic acid can be easily accomplished by applying the sample to a membrane and then UV-crosslinking. For example, the samples may be arranged in an array so that the array can be sequentially interrogated with one or more sets of PNA Kissing Probes to thereby determine whether each sample contains one or more target sequence of interest.

In still another embodiment, the fluorophore-labeled PNA Probe is immobilized to a support and the samples and the quencher-labeled PNA Probe are sequentially interrogated to thereby determine whether each sample contains a target sequence of interest. In preferred embodiments, fluorophore-labeled PNA Probe is immobilized on an array which is contacted with the sample of interest. Consequently, the sample can be simultaneously analyzed for the presence and quantity of numerous target sequences of interest wherein the composition of the fluorophore-labeled PNA Probe and quencher-labeled PNA probe are judiciously chosen and the fluorophore-labeled PNA Probe is arranged at predetermined locations on the surface so that the presence, absence or amount of particular target sequences can be unambiguously determined. Arrays of PNA Kissing Probes are particularly useful because no second detection system is required since PNA Kissing Probes are self-indicating. Consequently, this invention is also directed to an array comprising two or more sets of PNA Kissing Probes with the fluorophore-labeled PNA probes being support bound and suitable for detecting, identifying or quantitating a target sequence of interest.

h. Kits

In yet another embodiment, this invention is directed to kits suitable for performing an assay, which detects the presence, absence or amount of one or more target sequences, which may be present in a sample. The characteristics of PNA Kissing Probes suitable for the detection, identification and/or quantitation of amount of one or more target sequence have been previously described herein. Furthermore, methods suitable for using the PNA Kissing Probes components of a kit to detect, identify or quantitate one or more target sequences, which may be present in a sample, have also been previously described herein.

The kits of this invention comprise one or more sets of PNA Kissing Probes and other reagents or compositions, which are selected to perform an assay or otherwise simplify the performance of an assay. Preferred kits contain sets of PNA Kissing Probes, wherein each of at least two sets of PNA Kissing Probes are used to distinctly detect and distinguish between the two or more different target sequences which may be present in the sample. Thus, the sets of PNA Kissing Probes are preferably labeled with independently detectable fluorophores so that each of the two or more different target sequences can be individually detected, identified or quantitated (a multiplex assay).

i. Exemplary Applications for Using the Invention:

The PNA Kissing Probes, methods and kits of this invention are particularly useful for the detection, identification and/or quantitation of microorganisms in clinical samples, e.g. urine, blood, wounds, sputum, laryngeal swabs, gastric lavage, bronchial washings, biopsies, aspirates, expectorates as well as in food, beverages, water, pharmaceutical products, personal care products, dairy products or environmental samples and cultures thereof.

In another embodiment, the invention may be used to detect a target sequence which is indicative for a genetically based disease or is indicative for a predisposition to a genetically based disease. Non-limiting examples of diseases include, beta.-Thalassemia, sickle cell anemia, Factor-V Leiden, cystic fibrosis and cancer related targets such as p53, p10, BRC-1 and BRC-2.

In still another embodiment, the target sequence may be related to a chromosomal DNA, wherein the detection, identification or quantitation of the target sequence can be used in relation to forensic techniques such as prenatal screening, paternity testing, identity confirmation or crime investigation.

Having described the preferred embodiments of the invention, it will now become apparent to one of skill in the art that other embodiments incorporating the concepts described herein may be used. It is felt, therefore, that these embodiments should not be limited to disclosed embodiments but rather should be limited only by the spirit and scope of the following claims.

As discussed, the present invention provides a method for the analysis of a target sequence in a sample (sometimes called a “first” sample) which in one embodiment includes the steps of contacting the first sample with a mixture that includes a mixture of two probes (probe A and probe B), in which:

i) Probe A includes of a nucleotide sequence, directed to a region of the target sequence and is labeled with a fluorophore at the end which, upon hybridization is closest to the adjacent target region for Probe B; and
ii) Probe B includes a nucleotide sequence, which cohybridizes to a region of the target sequence adjacent to the target region of Probe A and is labeled with a quencher which, upon hybridization is closest to the adjacent target region for Probe A. Preferably, the method further includes a step b. involving measuring fluorescence following cohybridization of Probe A and Probe B to the target sequence, under suitable hybridization conditions, in which presence or amount of target sequence present in the first sample is negatively correlated with the fluorescence of the fluorophore on Probe A.

More particular Probe A embodiments for use with the forgoing method include those in which the probe is labeled with the quencher at or near the end of the probe ie., at one or more of the 5′ end, the 3′ end, or within five (5) nucleobases from said end, particularly one or two nucleobases from said end. In a particular embodiment, the quencher is bound to the 5′ or 3′ end of Probe A.

The foregoing particular invention method is flexible and can be adapted as needed to suit one or a combination of specific detection strategies. For instance, in one approach, the fluorescence (quenching) is measurably different than a suitable control. As used herein, “significantly different” or “measurably different” are used to describe experimental values obtained for fluorescence measurements which diverge meaningfully from a control, and within the precision of the measuring device. Differences in values are sometimes described using the statistical parameter of the standard deviation of the mean. A frequently used rule of thumb states that measurements which differ by more than two standard deviations from each other are considered statistically significant. A more stringent test of significance would require differences of three standard deviations, and so on. Ultimately, the judgment of what is defined as statistical significance depends on the individual criteria of a given test as defined by the precision and accuracy of the measuring device, and the error tolerance of the assay. Suitable controls for this invention application include, but are not limited to, a positive control, a negative control, a no target control or a second sample.

Measurement of relative quenching of a sample to gain information about its nature or sequence is also envisioned as aspect of the invention. Relative quenching is measured by comparison of the signal of a sample to a positive or negative control, a no target control, or a second sample. The determination of different amounts of quenching between samples may infer differences in target concentration, differences in target sequence, or differences in target state. Examples of differences in target sequence which may be inferred by relative quenching differences between samples include base insertions, base deletions, nucleotide polymorphisms, splice variants, mutations. Examples of differences in target state which may be inferred by relative quenching differences between samples include degradation, methylation, folding, hybridization, secondary structure, protein association. Examples of differences in target concentration which may be inferred by relative quenching differences between samples are self-evident, but may also be used to infer target concentrations prior to a particular manipulation, such as PCR amplification. Where target concentrations are fixed, or at least relatively close, for instance after a PCR reaction in which reaction components were exhausted, differences in relative quenching between samples may be of even greater diagnostic value. An example might include a mutant target which differs in sequence composition by a single base (as compared to the wild type target), inserted between the binding regions of otherwise immediately adjacent probes of this invention. Relative quenching differences of the amplified target compared to wild type control, could be diagnostic of the presence or absence of the inserted base due to the changes in quenching which occur as probe binding sites are separated in space by a single base. This aspect of the invention exceeds the present art by including information about the target due to the spatial orientation of the probes, as well as the presence or absence of the binding sites. Also, since two probes are required to observe quenching the specificity of the probes is essentially compounded.

In a more specific embodiment of the method, the fluorescence is taken to be indicative of an increase in target concentration, a structural difference between sample and control target sequences or a change in the state of the target sequence. Typically, the structural difference between sample and control target sequences will be at least one of the following: a nucleobase insertion, nucleobase deletion, genetic polymorphism, splice variation, amplification (eg., genetic or PCR amplification) or a mutation. One or a combination of target state changes are detectable by the invention including at least one of degradation, methylation, folding, hybridization, or association of the target sequence with protein.

In some applications of the method in which further target specificity is desired, it will be useful to pre-modify at least one of Probe A and Probe B. Thus, for instance, a user of the invention wanting to increase hybridization to target sequences in embodiments in which substantial non-target sequence is present (or suspected to be present) can within the scope of the present invention modify at least one of Probe A and/or Probe B to include at least one nucleobase differing from a corresponding unwanted DNA or RNA eg., change less than five (5), preferably less than four (4), and more typically about one (1), two (2) or three (3) of such nucleobases. Examples of non-target sequences include those having significant sequence similarity to desired target sequences as determined by conventional hybridization assays eg., Southern blotting, computer-assisted homology searches and related assays. More specific examples of non-target sequences include situations in which a sample to be analyzed has (or is suspected of having) one or more species closely related (ie. within the same genus or family) to the organism of interest.

The forgoing specific method can be used to analyse target sequence in nearly any application disclosed herein including detecting target sequence in a closed tube (homogeneous) assay; detecting a nucleic acid that includes a target sequence wherein said nucleic acid has been synthesized or amplified in a reaction occurring in the closed tube (homogeneous) assay. Examples of suitable nucleic acid synthesis or nucleic acid amplification reactions are known in the field and include, but are not limited to, Polymerase Chain Reaction (PCR), Ligase Chain Reaction (LCR), Strand Displacement Amplification (SDA), Transcription-Mediated Amplification (TMA), Rolling Circle Amplification (RCA) and Q beta replicase. A particularly useful PCR reaction to this invention is an asymmetric PCR reaction.

In some instances, it may be useful to adapt the forgoing method to provide even more target specificity ie. alone or in combination with modifying at least one of the Probe A and Probe B to avoid non-target sequences as discussed. In this embodiment, the method will further include contacting the sample with at least one blocking probe as described herein (eg., less than about four (4) or five (5) of such blocking probes, typically, one (1) or (2) of same) in which case the method will include the specific step of hybridizing the blocking probe to the unwanted DNA or RNA, if present (or suspected to be present) in the sample.

As discussed, the forgoing method can be used in one or a combination formats including detecting, identifying or qualifying the presence or amount of a microorganism in the sample. Typically, such a method is used to detect, identify, or quantitate the presence or amount of one or more species or types of a microorganism in the sample. Also, the method can, in some embodiments, be used to determine the effect of antimicrobial agents on the growth of one or more microorganisms in the sample. If desired, the method is also suitable for determining the presence or amount of a taxonomic group of microorganisms in the sample. The method is also suitable for diagnosing a condition of medical interest.

For use with the invention, the target sequence can be provided in a variety of formats including soluble, semi-soluble or forms bound (immobilized) to a surface. Examples of suitable surfaces include solid or semi-solid supports (eg., beads, resins, sols, gels, and the like). In these invention embodiments, the Probe A is immobilized to a surface and it may be one component of an array.

Suitable probes for use with the invention include those in which Probe A, Probe B or both consists of, consists essentially of or at least includes a monomer unit of PNA or LNA. Thus in particular invention embodiments, Probe A, Probe B, or both is fully PNA, LNA or a nucleic acid. Alternatively, Probe A, Probe B or both include at least one LNA monomer unit in additional to nucleic acid. In such an embodiment, “gapmers” or “chimeric” probes are contemplated for use with the invention such as those having the general formula: DNA-LNA-DNA as well as related probes.

Alternatively, or in addition, one or more of the blocking probes described herein consists consists essentially of or at least includes a monomer unit of PNA or LNA. Additional blocking probe embodiments include those in which the probe is fully PNA, LNA or nucleic acid. The blocking probe may be fully PNA, LNA or nucleic acid. Blocking probes with a “gapmer” or “chimeric” structure as mentioned previously for Probes A and B are also contemplated and within the scope of the present invention. Probes for use with the invention include those that are linear PNA beacons as defined herein.

Successful practice of the invention can be accomplished with one or a variety of samples. In one embodiment, the sample includes (or is suspected of having) a target sequence which is indicative of genetically based disease. Alternatively, a suitable target sequence is indicative of a predisposition to a genetically based disease. The invention is not limited to the analysis of any particular target sequence, however the following are specifically envisioned: target sequences associated with 5-Thalassemia, sickle cell anemia, Factor-V Leiden, cystic fibrosis and cancer related targets such as p53, pIO, BRC-1 and BRC-2. Sequence information for these and a variety of other suitable targets can be obtained from a variety of public databases including, but not limited to, the National Center for Biotechnology Information (NCBI)—Genetic Sequence Data Bank (Genbank), National Library of Medicine, 38A, 8N05, Rockville Pike, Bethesda, Md. 20894. See Benson, D. A. et al. (1997) Nucl. Acids. Res. 25: 1 for a description of Genbank. Particular target sequences of interest include those of forensic interest such as prenatal screening, paternity testing, identity confirmation or crime investigation.

As also discussed, the invention features an array that includes, in one embodiment, a probe A immobilized to a surface support in which cohybridization of pairs of Probe A and Probe B to adjacent target sequences at predetermined locations are suitable for analysis of two or more target sequences present in a sample. If desired, the sample further includes at least one blocking probe (eg., less than about four (4) or five (5) of such blocking probes, typically, one (1) or (2) of same). Suitable blocking probes for use with the array can be any of those previously mentioned.

A wide range of particular arrays are within the scope of the invention such as those suitable for regeneration by treatment with one or more regeneration catalysts selected from the group consisting of heat, nuclease enzyme and chemical denaturant such as aqueous solutions containing formamide, urea and/or sodium hydroxide.

### I. DEFINITIONS

a. As used herein, the term “nucleobase” means those naturally occurring and those non-naturally occurring heterocyclic moieties commonly known to those who utilize nucleic acid technology or utilize peptide nucleic acid technology to thereby generate polymers that can sequence specifically bind to nucleic acids.
b. As used herein, the term “nucleobase sequence” means any segment of a polymer that comprises nucleobase containing subunits. Non-limiting examples of suitable polymers or polymers segments include oligodeoxynucleotides, oligoribonucleotides, peptide nucleic acids, nucleic acid analogs, nucleic acid mimics, and/or chimeras. Suitable nucleic analogs for use with the probes of this invention can include at least one locked nucleic acid (LNA) subunit as described by Koshkin et al. Tett. Lett. 39: 4381 (1998); Koshkin et al. Tetrahedron 54: 3607 (1998); Arya et al. J. Amer. Chem. Soc. 120: 6619 (1998). See also U.S. Pat. Nos. 6,794,499; 6,670,461 and allowed U.S. application Ser. No. 10/208,650 as filed on Jul. 29, 2002 (disclosing how to make and use a wide variety of LNA compositions).
c. As used herein, the term “target sequence” means the nucleobase sequence that is to be detected by the probe. The term “target region” or “region of the target sequence” refers to a part of the target sequence. As used herein, the term “probe” means a polymer (e.g. a DNA, RNA, PNA, chimera or linked polymer) having a probing nucleobase sequence that is designed to sequence specifically hybridize to a target sequence of a target molecule of an organism of interest.
d. As used herein, the term “peptide nucleic acid” or “PNA” means any oligomer, linked polymer or chimeric oligomer, comprising two or more PNA subunits (residues), including any of the polymers referred to or claimed as peptide nucleic acids in U.S. Pat. Nos. 5,539,082, 5,527,675, 5,623,049, 5,714,331, 5,736,336, 5,773,571, 5,786,461, 5,837,459, 5,891,625, 5,972,610 and 5,986,053; all of which are herein incorporated by reference. The term “peptide nucleic acid” or “PNA” shall also apply to polymers comprising two or more subunits of those nucleic acid mimics described in the following publications: Diderichsen et al., Tett. Lett. 37: 475-478 (1996); Fujii et al., Bioorg. Med. Chem. Lett. 7: 637-627 (1997); Jordan et al., Bioorg. Med. Chem. Lett. 7: 687-690 (1997); Krotz et al., Tett. Lett. 36: 6941-6944 (1995); Lagriffoul et al., Bioorg. Med. Chem. Lett. 4: 1081-1082 (1994); Lowe et al., J. Chem. Soc. Perkin Trans. 1, (1997) 1: 539-546; Lowe et al., J. Chem. Soc. Perkin Trans. 11: 547-554 (1997); Lowe et al., J. Chem. Soc. Perkin Trans. 11: 555-560 (1997); Petersen et al., Bioorg. Med. Chem. Lett. 6: 793-796 (1996); Diederichsen, U., Bioorganic s Med. Che11Z. Lett., 8: 165168 (1998); Cantin et al., Tett. Lett., 38: 4211-4214 (1997); Ciapetti et al., Tetrahedron, 53: 1167-1176 (1997); Lagriffoule et al., Chem. Eslr. J., 3: 912-919 (1997) and the Peptide-Based Nucleic Acid Mimics (PENAMs) of Shah et al as disclosed in W096/04000.

In preferred embodiments, a PNA is a polymer comprising two or more subunits of the formula: wherein, each J is the same or different and is selected from the group consisting of H, RI, Oral, SURI, NHRI, NUL2, F, Cl, Br and I. Each K is the same or different and is selected from the group consisting of O, S, NH and NRI. Each RI is the same or different and is an alkyl group having one to five carbon atoms that may optionally contain a heteroatom or a substituted or unsubstituted aryl group. Each A is selected from the group consisting of a single bond, a group of the formula; —(CJ2)s- and a group of the formula; —(CJ2)sC(O)—, wherein, J is defined above and each s is an integer from one to five. The integer t is 1 or 2 and the integer u is 1 or 2. Each L is the same or different and is independently selected from the group consisting of J, adenine, cytosin, guanine, thymine, uridine, 5-methylcytosine, 2-aminopurine, 2-amino-6-chloropurine, 2,6-diaminopurine, hypoxanthine, pseudoisocytosine, 2-thiouracil, 2-thiothymidine, other naturally occurring nucleobase analogs, other non-naturally occurring nucleobases, substituted and unsubstituted aromatic moieties, biotin, fluorescein and dabcyl.

In the most preferred embodiment, a PNA subunit consists of a naturally occurring or non-naturally occurring nucleobase attached to the aza nitrogen of the N-[2-(aminoethyl)] glycine backbone through a methylene carbonyl linkage.

g. As used herein, the terms “label” and “detectable moiety” are interchangeable and shall refer to moieties that can be attached to a probe to thereby render the probe detectable by an instrument or method. h. As used herein, the term “chimera” or “chimeric oligomer” means an oligomer comprising two or more linked subunits that are selected from different classes of subunits.

For example, a PNA/DNA chimera would comprise at least two PNA subunits linked to at least one 2′-deoxyribonucleic acid subunit (For exemplary methods and compositions related to PNA/DNA chimera preparation See: W096/40709). Exemplary component submits of the chimera are selected from the group consisting of PNA subunits, naturally and non-naturally occurring amino acid subunits, DNA subunits, RNA subunits and subunits of analogues or mimics of nucleic acids. i. As used herein, the term “linked polymer” means a polymer comprising two or more polymer segments that are linked by a linker. The polymer segments that are linked to form the linked polymer are selected from the group consisting of an oligodeoxynucleotide (DNA), an oligoribonucleotide (RNA), a peptide, a polyamide, a peptide nucleic acid (PNA) and a chimera.

### 2. Description

### I. General:

### PNA Synthesis:

Methods for the chemical assembly of PNAs are well known (See: U.S. Pat. Nos. 5,539,082, 5,527,675, 5,623,049, 5,714,331, 5,736,336, 5,773,571, 5,786,461, 5,837,459, 5,891,625, 5,972,610 and 5,986,053; all of which are herein incorporated by reference.

Chemicals and instrumentation for the support bound automated chemical assembly of peptide nucleic acids are now commercially available. Both labeled and unlabeled PNA oligomers are likewise available from commercial vendors of custom PNA oligomer.

Chemical assembly of a PNA is analogous to solid phase peptide synthesis, wherein at each cycle of assembly the oligomer possesses a reactive alkyl amino terminus that is condensed with the next synthon to be added to the growing polymer. Because standard peptide chemistry is utilized, natural and non-natural amino acids are routinely incorporated into a PNA oligomer.

Because a PNA is a polyamide, it has a C-terminus (carboxyl terminus) and an N-terminus (amino terminus). For the purposes of the design of a hybridization probe suitable for antiparallel binding to the target sequence (the preferred orientation), the N-terminus of the probing nucleobase sequence of the PNA probe is the equivalent of the 5′hydroxyl terminus of an equivalent DNA or RNA oligonucleotide.

### PNA Labeling:

Preferred non-limiting methods for labeling PNAs are described in U.S. Pat. No. 6,110,676, W099/22018, W099/21881, W099/49293 and W099/37670, the examples section of this specification or are otherwise well known in the art of PNA synthesis and peptide synthesis.

### Labels:

Fluorophores are the detectable moieties suitable for labeling PNA Kissing Probes of this invention. Preferred fluorochromes (fluorophores) include 5 (6)-carboxyfluorescein (Flu), 6-((7-amino-4-methylcoumarin-3-acetyl) amino) hexanoic acid (Cou), 5 (and 6)-carboxy-X-rhodamine (Rox), Cyanine 2 (Cy2) Dye, Cyanine 3 (Cy3) Dye, Cyanine 3.5 (Cy3.5) Dye, Cyanine 5 (Cy5) Dye, Cyanine 5.5 (Cy5.5) Dye Cyanine 7 (Cy7) Dye, Cyanine 9 (Cy9) Dye (Cyanine dyes 2, 3, 3.5, 5 and 5.5 are available as NHS esters from Amersham, Arlington Heights, Ill.), JOE, Tamara or the Alexa dye series (Molecular Probes, Eugene, Oreg.).

Fluorescence quenchers are the acceptor moieties suitable for labeling PNA Kissing Probes of this invention.

The preferred quencher is 4-((-4(dimethylamino)phenyl)azo)benzoic acid (dabcyl).

### Detectors:

Other suitable labeling reagents and preferred methods of attachment would be recognized by those of ordinary skill in the art of PNA, peptide or nucleic acid synthesis.

### EXAMPLES

TABLE 1

| | | |
|---|---|---|
| Type | ID | Sequence (5′-3′) |

| | | |
|---|---|---|
| PNA | F1 | Flu-OO-GCTTCTCGTCCGTTC |
| PNA | F2 | Flu-OO-ACACCAAACCTCAGC |
| DNA | F3 | Flu-ACACCAAACCTCAGCGCT |
| PNA | Q1 | ACTTCAAAGGAGCAA-K-K(Dabcyl) |
| DNA | Q2 | ATCACTTCAAAGGAGCAA-K-K(Dabcyl) |
| DNA | D1 | AGCGAACGGACGAGAAGCTTGCTTCTCTGATCTTAG |
| DNA | D2 | AGCGCTGAGGTTTGGTGTTTGCTCCTTTGAAGTTAG |
| DNA | D3 | AGCGCTGAAATTTGGTGTTTGCTCCTTTGAAGTTAG |

With reference to Table 1, the table displays the probes and targets used in the following experiments. The following abbreviations are used; Flu=fluorescein, O=O linker, K=lysine. The underlined region of DNA target D1 denotes the area where PNA probes F1 and Q1 are substantially complementary. The underlined region of DNA targets D2 and D3 denote the area where PNA probes F2 and Q1 are substantially complementary. Note however that the targets D2 and D3 differ by two bases, and that PNA probe F2 is perfectly complementary to target D2. DNA probe F3 is substantially complementary to the same region of D2 as PNA F2, but is three bases longer. Likewise, Q2 is the same sequence as the Q1 plus three bases, and Q1 is a PNA probe, and Q2 is DNA.

### Example 1

### Detection of DNA Targets by Signal Quenching

PNA probes F1, Q1 and a series of dilutions of DNA targets D1 were made in 10 mM Tris pH 10.0, 30% formamide such that the final concentration of each PNA probe was 25 nM, and the concentration of DNA target was varied. The DNA target concentration ranged from 0.0 nM to 50.0 nM. 2 mL aliquots of each probe/target dilution was placed in an optically clear cuvette and allowed to equilibrate at room temperature for 30 minutes. Duplicate fluorescence measurements of each sample were made on a Turner TD-700 fluorometer equipped to measure fluorescent signal from fluorescein.

With reference to Table 2, the table displays DNA target, D1, concentration in column 1, and fluorescent signal using various probe mixtures in columns 2, and 3. D1 concentrations tested range from 0.0 nM to 50 nM. Average fluorescence measured at 0.0 nM of DNA target indicates the “baseline” fluorescence. Any measurement which differs substantially below the baseline fluorescence indicates quenching due to hybridization of both probes F1 and Q1. As can be seen in Table 2, column 2, the fluorescent signal is not substantially less than the baseline fluorescence at 1.3 nM, 2.5 nM and 5.0 nM. At 12.5 nM D1 fluorescent signal is observed to decrease, and at higher, concentrations of D1, the signal continues to decrease. These data demonstrate that the sensitivity of this particular system to detect DNA target, D1, is at or below 12.5 nM.

TABLE 2

| | 1 | 2 |
|---|---|---|
| A | [Target] | F1/Q1/D1 |
| B | 0.0 nM | 349.4 |
| C | 1.3 nM | 375.0 |
| D | 2.5 nM | 369.0 |
| E | 5.0 nM | 369.4 |
| F | 12.5 nM | 322.5 |
| G | 25.0 nM | 272.4 |
| H | 50.0 nM | 223.2 |

### Example 2

### Detection of Specific DNA Targets by Signal Quenching

A series of dilutions of PNA probes F2, Q1 and DNA targets D2 and D3 was made in 10 mM Tris pH 10.0, 50% formamide such that the final concentration of each PNA probe was 25 nM, and the concentration of DNA target was varied. The DNA target concentration ranged from 0.0 nM to 25.0 nM. The DNA probe, F3 was also tested, though at 30% formamide, and with 200 mM NaCl, 10 mM Tris pH 10.0. 2 mL aliquots of each probe/quencher/target dilution were placed in an optically clear cuvettes and allowed to equilibrate at room temperature for 30 minutes. Duplicate fluorescence measurements of each sample were made on a Turner TD-700 fluorometer equipped to measure fluorescent signal from fluorescein.

With reference to Table 3, the table displays DNA target concentration in column 1, and average fluorescent signals for various probe/quencher/target combinations in columns 2, 3 and 4. D2 and D3 concentrations tested range from 0.0 nM to 25.0 nM. Average fluorescence measured at 0.0 nM of DNA target indicates the “baseline” fluorescence. Any measurement which differs substantially below the baseline fluorescence indicates quenching due to hybridization of both the fluorescent labeled and the quencher labeled probes.

TABLE 3

| | 1 | 2 | 3 | 4 |
|---|---|---|---|---|
| A | [Target] | F2/Q1/D2 | F2/Q1/D3 | F3/Q1/D2 |
| B | 0.0 nM | 490.3 | 490.3 | 501.0 |
| C | 1.3 nM | 467.6 | 484.8 | — |
| D | 2.5 nM | 461.7 | 489.3 | — |
| E | 5.0 nM | 424.5 | 491.1 | 401.5 |
| F | 12.5 nM | 353.5 | 491.5 | — |
| G | 25.0 nM | 207.7 | 494.3 | 326.6 |

As can be seen in column 2 of Table 3, the fluorescent signal from DNA target, D2, begins to differ significantly from the no target control (0.0 nM) at the lowest target concentration, 1.3 nM. The signal continues to decrease as more target, D2, is added, until the smallest fluorescent value is seen at 25 nM D2. Hence the quenching is related to the target concentration. Conversely, addition of the mismatched target, D3, does not correlate to a decrease in signal as can be seen in column 3. With reference to Table 3, column 4, though measurements were only made at 5.0 nM and 25 nM D2 target, there is clearly a negative correlation between target concentration and fluorescence when a labeled DNA probe is used in conjunction with a PNA quencher probe.

### Example 3

### Comparison of PNA and DNA Quencher Probes

A series of dilutions of DNA probe F3, either Q1 or Q2, and DNA targets D2 and D3 was made in 200 mM NaCl, 10 mM Tris pH 10.0, 30% formamide such that the final concentration of each probe was 25 nM, and the concentration of DNA target was varied. The DNA target concentrations were set to 0.0 nM (no target), 12.5 nM, or 25.0 nM. 2 mL aliquots of each probe/quencher/target dilution were placed in optically clear cuvettes and allowed to equilibrate at room temperature for 15 minutes. Duplicate fluorescence measurements of each sample were made on a Turner TD-700 fluorometer equipped to measure fluorescent signal from fluorescein.

With reference to Table 4, the table displays DNA target concentration in column 1, and average fluorescent signals for various probe/quencher/target combinations in columns 2, 3, 4 and 5. Average fluorescence measured at 0.0 nM of DNA target indicates the “baseline” fluorescence. Any measurement which differs substantially below the baseline fluorescence indicates quenching due to hybridization of both the fluorescent labeled and the quencher labeled probes.

TABLE 4

| | 1 | 2 | 3 | 4 | 5 |
|---|---|---|---|---|---|
| A | [Target] | Q2/D2 | Q2/D3 | Q1/D2 | Q1/D3 |
| B | 00.0 nM | 522 | 522 | 512 | 512 |
| C | 12.5 nM | 400 | 514 | 296 | 455 |
| D | 25.0 nM | 346 | 500 | 187 | 437 |

As can be seen in column 2 of Table 4, the fluorescent signal from DNA target, D2, begins to differ significantly from the no target control (0.0 nM) at the lowest target concentration, 12.5 nM and continues to decrease at 25 nM. A similar, though more dramatic decrease is observed in column 4 using the same probe and target, and the PNA quencher probe. In the case of the mismatched target (D3) both the DNA and PNA quencher probe samples (columns 3 and 5 respectively) show a slight decrease in signal as the DNA probe, F3, weakly binds to the mismatch. These data demonstrate that combinations of PNA and DNA probes are useful embodiments of this invention. Though both useful, this example demonstrates that the PNA/DNA probe set is preferred over the DNA/DNA probe set, because of PNA's improved hybridization properties.

This invention has been described in detail with reference to preferred embodiments thereof. However, it will be appreciated that those skilled in the art, upon consideration of this disclosure, may make modifications and improvements within the spirit and scope of the invention.

All references disclosed herein are incorporated by reference.

What is claimed is:

1. A method for the analysis of a target sequence in a first sample,
said method comprising:
b. contacting the first sample with a mixture comprising:
A mixture of two probes (probe A and probe B), wherein:
i) Probe A is comprised of a nucleotide sequence, directed to a region of the target sequence and is labeled with a fluorophore at the end which, upon hybridization is closest to the adjacent target region for Probe B; and
ii) Probe B is comprised of a nucleotide sequence, which cohybridizes to a region of the target sequence adjacent to the target region of Probe A and is labeled with a quencher which, upon hybridization is closest to the adjacent target region for Probe A,
b. measuring fluorescence following cohybridization of Probe A and Probe B to the target sequence, under suitable hybridization conditions, wherein the presence or amount of target sequence present in the first sample can be negatively correlated with the fluorescence of the fluorophore on Probe A.

2. The method of claim 1, wherein the fluorescence is measurably different than a control.

3. The method of claim 2, wherein the control is a positive control, a negative control, a no target control or a second sample.

4. The method of claim 3, wherein the fluorescence is taken to be indicative of an increase in target concentration, a structural difference between sample and control target sequences or a change in the state of the target sequence.

5. The method of claim 4, wherein the structural difference between sample and control target sequences is at least one of a nucleobase insertion, nucleobase deletion, genetic polymorphism, splice variation, amplification or mutation.

6. The method of claim 4, wherein the change in target state is due to at least one of degradation, methylation, folding, hybridization, or association of the target sequence with protein.

7. The method of claim 1, wherein the target sequence for Probe A and Probe B comprise at least one nucleobase differing from non-target sequences.

8. The method of claim 7, wherein the method is used to detect target sequence in a closed tube (homogeneous) assay.

9. The method of claim 7, wherein the method is used to detect a nucleic acid comprising a target sequence wherein said nucleic acid has been synthesized or amplified in a reaction occurring in the closed tube (homogeneous) assay.

10. The method of claim 9, wherein preferred nucleic acid synthesis or nucleic acid amplification reactions are selected from the group consisting of: Polymerase Chain Reaction (PCR), Ligase Chain Reaction (LCR), Strand Displacement Amplification (SDA), Transcription-Mediated Amplification (TMA), Rolling Circle Amplification (RCA) and Q beta replicase.

11. The method of claim 10, wherein the PCR reaction is an asymmetric PCR reaction.

12. The method of claim 1, wherein the method further comprises contacting the sample with at least one blocking probe.

13. The method of claim 12, wherein the method further comprises hybridizing the blocking probe to non-target sequences.

14. The method of claim 1, wherein the method further comprises detecting, identifying or qualifying the presence or amount of a microorganism in the sample.

15. The method of claim 1, wherein the method is used to detect, identify, or quantitate the presence or amount of one or more species or types of a microorganism in the sample.

16. The method of claim 1, wherein the method is used to determine the effect of antimicrobial agents on the growth of one or more microorganisms in the sample.

17. The method of claim 1, wherein the method is used to determine the presence or amount of a taxonomic group of microorganisms in the sample.

18. The method of claim 1, wherein the method is used to diagnose a condition of medical interest.

19. The method of claim 1, wherein the target sequence is immobilized to a surface.

20. The method of claim 1, wherein Probe A is immobilized to a surface.

21. The method of claim 1, wherein Probe A is one component of an array.

22. The method of claim 1, wherein Probe A is PNA or LNA.

23. The method of claim 1 or 22, wherein Probe B is a PNA or LNA.

24. The method of claim 1, wherein Probe A is a nucleic acid comprising LNA.

25. The method of claim 1 or 24, wherein Probe B is a nucleic acid comprising LNA.

26. The method of claim 12, wherein the blocking probe is PNA or LNA.

27. The method of claim 12, wherein the blocking probe is a nucleic acid comprising LNA.

28. The method of claim 1, wherein at least one of the probes is a linear PNA beacon.

29. The method of claim 1, wherein the sample comprises a target sequence which is indicative for a genetically based disease or is indicative for a predisposition to a genetically based disease

30. The method of claim 1, wherein the target sequence is associated with a disease selected from the group consisting of 5-Thalassemia, sickle cell anemia, Factor-V Leiden, cystic fibrosis and cancer related targets such as p53, pIO, BRC-1 and BRC-2.

31. The method of claim 1, wherein the target sequence in a forensic technique such as prenatal screening, paternity testing, identity confirmation or crime investigation.

32. An array comprising a probe A immobilized to a surface support, wherein cohybridization of pairs of Probe A and Probe B to adjacent target sequences at predetermined locations are suitable for analysis of two or more target sequences present in a sample.

33. The array of claim 32, wherein the sample further comprises at least one blocking probe.

34. The array of claim 32, wherein the array is suitable for regeneration by treatment with one or more regeneration catalyst selected from the group consisting of heat, nuclease enzyme and chemical denaturant such as aqueous solutions containing formamide, urea and or sodium hydroxide.

35. The array of claim 32, wherein Probe A is PNA or LNA.

36. The array of claim 32 or 35, wherein Probe B is a PNA or LNA.

37. The array of claim 32, wherein Probe A is a nucleic acid comprising LNA.

38. The array of claim 32 or 37, wherein Probe B is a nucleic acid comprising LNA.

39. The array of claim 33, wherein the blocking probe is PNA or LNA.

40. The array of claim 33, wherein the blocking probe is a nucleic acid comprising LNA.

41. The array of claim 32, wherein at least one of the PNA probes is a linear PNA beacon.
